(19) United States
(12) Patent Application Publication
(10) Pub. No.: US 2026/0219246 A1
(43) Pub. Date: Jul. 30, 2026

(54) METHODOLOGY TO GENERATE DECOUPLED REAL FUEL SURROGATES FOR 3-DIMENSIONAL SPRAY AND COMBUSTION MODELING IN ENGINE APPLICATIONS

(71) Applicant: SAUDI ARABIAN OIL COMPANY, Dhahran (SA)

(72) Inventors: Ji-Woong Park, Sterling Heights, MI (US); Anqi Zhang, Canton, MI (US)

(73) Assignee: ARAMCO SERVICES COMPANY, Hoston, TX (US)

(21) Appl. No.: 19/040,178

(22) Filed: Jan. 29, 2025

(51) Int. Cl. G01N 30/88; G01N 30/02; G01N 33/22; G01N 33/28

(52) U.S. Cl. CPC G01N 30/88; G01N 2030/025; G01N 2030/8854; G01N 33/225; G01N 33/2829

(57) ABSTRACT

Methods and systems are disclosed. The method includes determining, using a gas chromatography device communicably coupled to a computing device, a plurality of gas and liquid properties of a sample fuel, and receiving, with a processor of the computing device, a plurality of fuel targets defining desired combustion parameters of the surrogate fuel. The method further includes assigning, with the processor, a palette of liquid and gas fuel species based on the plurality of liquid and gas properties of the sample fuel, and determining, with the processor, a liquid and a gas surrogate formulation from the palette of liquid and gas fuel species. The method further includes outputting, with the processor, a proportional composition of chemical elements forming the surrogate fuel based on the liquid and gas surrogate formulation, and formulating the surrogate fuel with a gas processing facility based on the proportional composition of the chemical elements.

### BACKGROUND

Real fuels used in transportation applications may contain mixtures of hundreds to thousands of different hydrocarbon species. The large number of hydrocarbon species may impose challenges for numerical simulations of the applications due to complex input characterization and costly computations. As a result, many engineering applications rely on surrogate fuel methods to represent the physical and chemical behavior of real fuels, which may simplify computations. However, traditional methods to formulate surrogate fuels cannot capture the effects of compositional variations in the real fuels. Accordingly, it is desirable to obtain surrogate fuel formulations suitable to predict the effects of injection conditions, physical and chemical fuel properties, and compositional variations affecting fuel spray and combustion.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

Embodiments disclosed herein generally relate to a method to determine a composition of a surrogate fuel. The method includes determining, using a gas chromatography device communicably coupled to a computing device, a plurality of gas properties and a plurality of liquid properties of a sample fuel. The method further includes receiving, with a processor of the computing device, a plurality of fuel targets defining desired combustion parameters of the surrogate fuel. The method further includes assigning, with the processor of the computing device, a palette of liquid fuel species based on the plurality of liquid properties of the sample fuel, and assigning a palette of gas fuel species based on the plurality of gas properties of the sample fuel. The method further includes determining, with the processor of the computing device, a liquid surrogate formulation from the palette of liquid fuel species and determining a gas surrogate formulation from the palette of gas fuel species. The method further includes outputting, with the processor of the computing device, a proportional composition of chemical elements forming the surrogate fuel based on the liquid surrogate formulation and the gas surrogate formulation. The method further includes formulating the surrogate fuel with a gas processing facility based on the proportional composition of the chemical elements.

Embodiments disclosed herein also generally relate to a system that determines a composition of a surrogate fuel. The system includes a computing device. The computing device includes a processor configured to execute instructions and a memory configured to store the instructions. The system further includes a gas chromatography device communicably coupled to the computing device and configured to determine a plurality of gas properties and a plurality of liquid properties of a sample fuel. The processor is configured to receive a plurality of fuel targets defining desired combustion parameters of the surrogate fuel. The processor is further configured to assign a palette of liquid fuel species based on the plurality of liquid properties of the sample fuel and assign a palette of gas fuel species based on the plurality of gas properties of the sample fuel. The processor is further configured to determine a liquid surrogate formulation from the palette of liquid fuel species and to determine a gas surrogate formulation from the palette of gas fuel species. The processor is further configured to output a proportional composition of chemical elements forming the surrogate fuel based on the liquid surrogate formulation and the gas surrogate formulation. The system further includes a gas processing facility configured to formulate the surrogate fuel based on the proportional composition of the chemical elements.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1 depicts a gas chromatography device in accordance with one or more embodiments.

FIG. 2 depicts a system in accordance with one or more embodiments.

FIG. 3 depicts a surrogate fuel algorithm in accordance with one or more embodiments.

FIG. 4 depicts a lookup table in accordance with one or more embodiments.

FIG. 5 depicts a species-palette and palette-surrogate assignment process in accordance with one or more embodiments.

FIG. 6 depicts surrogate fuel candidates in accordance with one or more embodiments.

FIG. 7 depicts a flowchart in accordance with one or more embodiments.

FIG. 8 depicts a computing system in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that the singular forms “a,” “an,” and “the” include plural referents unless the context clearly dictates otherwise. For example, a “palette” may include any number of “palettes” without limitation. Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

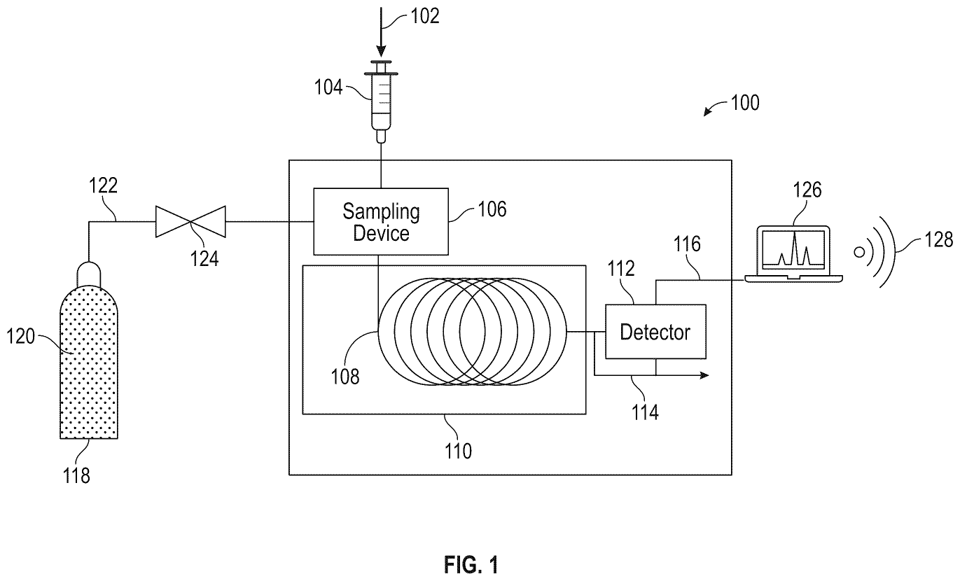

In the following description of FIGS. 1-8, any component described with regard to a figure, in various embodiments disclosed herein, may be equivalent to one or more like-named components described with regard to any other figure. For brevity, descriptions of these components will not be repeated with regard to each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described with regard to a corresponding like-named component in any other figure.

Real fuels such as gasoline, diesel, or jet fuel are highly complex mixtures containing hundreds of different hydrocarbon compounds. These compounds vary in molecular structure, size, and properties, leading to complex behaviors during combustion. As such, this high number of compounds present in real fuels makes it difficult to model or simulate the behavior of real fuels with enough accuracy. Accordingly, surrogate fuels simplify this complex task by using fewer, carefully selected chemical compounds that together mimic the key properties and performance of the real fuel. Therefore, the goal of the surrogate fuel is to replicate the key physical and chemical properties of the real fuel-such as ignition delay times, combustion characteristics such as flame region, and flame luminosity-without the complexity of its many components. In addition, surrogate fuels enable the development and validation of accurate predictive models of fuel combustion because they are easier to describe mathematically, yet still behave similarly to real fuels.

In general, the composition of real fuels may vary depending on crude oil sources, refining processes, and local fuel standards. For example, in the United States, gasoline may contain up to 10% ethanol as a result of federal regulations to reduce greenhouse gas emissions. In contrast, other countries may use different biofuels or have no ethanol requirements, thus leading to differences in gasoline composition. These compositional variations have an impact on vehicle performance by influencing fuel spray and combustion properties. As such, accurate predictive models of fuel composition are desirable for engine design and optimization. For example, an engine optimized for gasoline from the United States might not perform as well on gasoline from a region with different specifications and compositions.

However, traditional numerical simulations used to determine the composition of a surrogate fuel often model the liquid and gas sub-surrogates differently due to the significant differences in compressibility. As a result, two sub-surrogate models are typically needed for the liquid and gas phases, respectively, and they are intrinsically decoupled. On the other hand, fuel spray behavior is predominantly affected by the physical properties of the liquid phase fuel, while the gas phase fuel influences combustion through chemical kinetics. Thus, in order to properly model fuel-related processes in combustion engines, it is desirable to have different requirements for liquid and gas sub-surrogates. In other words, it is desirable that the liquid sub-surrogate focuses on spray-affecting physical properties, while the gas sub-surrogate closely follows the reactivity characteristics of the hydrocarbons.

Accordingly, embodiments disclosed herein generally relate to methods and systems for determining an optimal surrogate fuel composition. Specifically, the methods and systems in this disclosure use a surrogate fuel formulation approach suitable for 3-dimensional modeling of engine fuel spray and combustion. The surrogate fuel formulation approach is described in greater detail later in the instant disclosure. However, for now it is sufficient to state that in the present disclosure the surrogate fuel comprises of a liquid sub-surrogate and a gas sub-surrogate. Decoupling the models for each phase (i.e., gas and liquid) while finding an optimal surrogate fuel composition is important to achieve the most balanced benefits across fuel property accuracy and computational cost. In one or more embodiments, mass conservation is the primary link between the two sub-surrogates. Additionally, as will be described, a surrogate fuel algorithm assigns palette species to the liquid and gas sub-surrogates and optimizes the combination of species such that they reproduce the physical and chemical properties of the real fuel. The optimization process may be performed iteratively until a user-defined termination criterion is met. The output of the surrogate fuel algorithm is an optimal surrogate fuel composition, which is then formulated in a gas processing facility, and combusted in a contained manner in an engine such that the combustion reaction generates power. Accordingly, using these methods and systems, accurate fuel surrogates that are representative of real hydrocarbon fuels may be generated in a computationally-efficient manner. Depictions of various configurations of the system used to determine the optimal surrogate fuel composition and methods of its use are provided in FIGS. 1-8, along with accompanying descriptions.

FIG. 1 shows a chromatography device in accordance with one or more embodiments. Specifically, FIG. 1 shows a gas chromatography (GC) device (100). Chromatography is a laboratory technique used for the separation of a mixture into its individual components. It is widely used in analytical chemistry for the identification and quantification of compounds. The technique is based on the principle that different compounds will move through a medium (a stationary phase) at different rates when carried by a fluid (a mobile phase), thus leading to their separation. It is noted that many types of chromatography techniques exist, such as paper chromatography, thin layer chromatography, column chromatography, and high-performance liquid chromatography, among others. Therefore, one with ordinary skill in the art will recognize that any type of chromatography devices and techniques may be employed without departing from the scope of this disclosure. Further, it is emphasized that the following discussions of chromatography are basic summaries and should not be considered limiting.

As noted, a GC device (100) is an analytical instrument used to separate, detect, and analyze the chemical components of compounds that may be vaporized without decomposition. GC uses a gas as the mobile phase and a liquid or solid as the stationary phase and it is particularly useful for studying volatile compounds. This is because volatile compounds have a relatively low boiling point and may therefore be easily vaporized and converted into gas. This efficient vaporization allows for volatile compounds to travel through the medium (i.e., the stationary phase) with the carrier gas (i.e., the mobile phase). As such, the separation of compounds in GC is highly efficient, especially for small, volatile molecules. In addition, volatile compounds are typically thermally stable at the temperatures used in GC, which ensures that the compounds do not decompose during the analysis, thus leading to accurate results.

A GC device (100) typically comprises several components designed to analyze mixtures of components. As shown in FIG. 1, a GC device (100) includes a sample (102), a syringe (104), a sampling device (106), a column (108), a column oven (110), a detector (112), a waste port (114), a first data connection (116), a storage tank (118), a carrier gas (120), an inlet line (122), a gas regulator (124), a first computing device (126), and a second data connection (128). Each of these components is subsequently described.

In general, a GC device (100) may analyze a broad range of samples (102), including, but not limited to, hydrocarbons, solvents, flavors, fragrances, and gases. In accordance with one or more embodiments, the sample (102) is a fuel sample. The fuel sample (102) is introduced in the GC device (100) using a syringe (104). A sampling device (106) vaporizes the liquid sample upon injection and controls the amount of sample entering the column (108). Typically, the fuel sample (102) is injected using a split mode or a split-less mode of injection. In the split mode, only a portion of the fuel sample (102) enters the column (108), while in the split-less mode, the entire fuel sample (102) is introduced. The vaporized sample is then mixed with the carrier gas (120) (i.e., the mobile phase).

Keeping with FIG. 1, the carrier gas (120) flows continuously and does not interact with the stationary phase, serving as the medium that transports the fuel sample (102) through the column (108). The carrier gas (120) is typically stored in a storage tank (118) (e.g., a high-pressure cylinder) and may be an inert gas. Examples of carrier gases (120) include, but are not limited to, Helium (H), Nitrogen (N), Argon (Ar), and Hydrogen (H). An inlet line (122) transport the carrier gas (120) from the storage tank (118) to the GC device (100). A gas regulator (124) controls the high pressure of the carrier gas (120) coming from the storage tank (118) such that it reaches a lower, usable pressure that is suitable for use in the GC device (100).

The column (108) is the component where the actual separation of compounds in the fuel sample (102) mixture takes place. Compounds in the fuel sample (102) interact with the stationary phase to different extents based on their physical and chemical properties. The stationary phase may be, for example, a thin film of liquid or solid on the inner surface of the column (108).

The column (108) comprises of a long tube (typically coiled) and is made of materials such as fused silica, stainless steel, or glass. The column (108) may be classified as a capillary column or a packed column. Capillary columns (108) are thin, flexible tubes made of fused silica. They are usually between 0.1 to 1 mm in internal diameter and may be up to 100 meters long. A liquid stationary phase is common in capillary columns (108), and it may be made of various polymers or silicones tailored for specific compound separations (e.g., polar or non-polar). On the other hand, packed columns (108) are thicker tubes (typically made of stainless steel or glass) filled with a solid stationary phase or a solid support that is coated with a liquid stationary phase. For example, the solid stationary phase may be a solid adsorbent such as silica gel, alumina, or porous polymers. Packed columns (108) are generally 1.5 to 10 mm in internal diameter and up to 3 meters long. Capillary columns (108) provide high resolution due to their narrow diameter and long length, while packed columns (108) are suitable for larger sample volumes or for separating gases. One with ordinary skill in the art will appreciate that many types of stationary phases exist and the fact that they are not enumerated herein does not impose a limit on the present disclosure.

The column (108) is housed in a column oven (110) which controls the temperature of the column (108) using a heater, thus ensuring an optimal separation of compounds. Typically, the column oven (110) temperature is gradually ramped (i.e., increased) during operation using a temperature controller (not shown). The temperature controller may allow temperature ramps from an initial temperature (e.g., 30-100° C.) to a final temperature (e.g., 250-300° C.) to optimize the separation process. Lower initial temperatures (e.g., 30° C. to 50° C.) may be used for volatile compounds to ensure they remain in the gas phase, while higher initial temperatures (e.g., 80° C. to 100° C.) may be used for less volatile compounds or for methods requiring a faster start. Higher final temperatures (e.g., 300° C. to 350° C.) may be used for high-boiling-point compounds or to ensure that the fuel sample (102) is completely evaporated from the column (108) at the end of the ramp.

Continuing with FIG. 1, the fuel sample (102) components that have been separated within the column (108) exit the column (108) sequentially and are directed toward the detector (112). As noted, the compounds in the fuel sample (102) mixture are separated based on their interactions with the stationary phase inside the column (108) and their volatility. These compounds elute (i.e., exit) the column at different times, known as their retention times. The carrier gas (120) that transports the sample through the column (108) also exits the column (108). At any given point, the effluent exiting the column (108) is a mixture of the eluted compounds and the carrier gas (120).

In accordance with one or more embodiments, the separated compounds are then detected by the detector (112). The detector (112) produces a signal proportional to the amount of each compound at that point in time, which is then recorded and analyzed using a first computing device (126) communicably coupled to the detector (112) using a first data connection (116). A waste port (114) diverts unwanted portions of the effluent, carrier gas (120), or contaminants away from the detector (112) and out of the GC device (100).

In accordance with one or more embodiments, the detector (112) may be a flame ionization (FI) detector, a thermal conductivity (TC) detector, or a mass spectrometer. One with ordinary skill in the art will appreciate that many types of detectors (112) exist, each suited to specific applications and types of compounds, and it will be understood that the type of detector (112) is selected by a system manufacturer based upon the contemplated sample composition. In addition, each of these detectors (112) have different principles of operation. While a full description of the principle of operation of the detector (112) exceeds the scope of this disclosure, a cursory introduction is provided herein.

A FI detector (112) measures ions produced during the combustion of organic compounds in a hydrogen-air flame. More specifically, the effluent exiting the column (108) and entering the FI detector (112) is mixed with hydrogen/air and ignited. The ignition of the mixture produces ions, which are collected by a pair of electrodes, thus generating an electrical current proportional to the amount of hydrocarbons in the fuel sample (102). A TC detector (112) measures changes in the thermal conductivity of the carrier gas (120) caused by the presence of different compounds. A TC detector (112) typically comprise of a filament and an electrical circuit. As the effluent exits the column (108) and passes over the heated filament, the filament temperature and its electrical resistance are modified by the presence of eluted compounds, which, in turn, affect the thermal conductivity of the carrier gas (120). These changes in the electrical resistance of the filament are detected by the electrical circuit (e.g., a Wheatstone bridge). A mass spectrometer provides detailed molecular information based on mass-to-charge ratios, allowing for compound identification. A mass spectrometer typically comprise of an ion source, a mass analyzer, and a detector (112). The effluent eluting from the column is ionized by the ion source, and the ions are separated by the mass analyzer based on their mass-to-charge ratio. The detector (112) (e.g., an electron multiplier) records the abundance of each ion and creates a mass spectrum.

In accordance with one or more embodiments, the signal produced by the detector (112) is recorded and analyzed using a first computing device (126). A chromatogram (i.e., a graphical representation of the detector's response as a function of time) is shown on the screen of the first computing device (126) in FIG. 1. A chromatogram displays the results of a chromatographic separation, showing how the separated compounds elute from the column (108) over time. Peaks in the chromatogram corresponds to the different compounds in the sample mixture. Further, the area under the peak is proportional to the quantity of the compound present. Accordingly, the liquid and gas properties of the fuel sample (102) may be determined using the GC device (100).

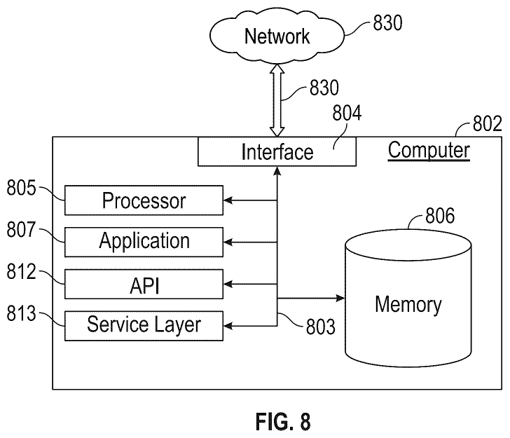

Keeping with FIG. 1, the first computing device (126) comprises or is functionally similar to that of a computer system discussed in greater detail in relation to FIG. 8 and the accompanying description. The first computing device (126) may include a memory and a processor that respectively serve to store and execute computer readable instructions. In addition, the first computing device (126) may include a Human Machine Interface (HMI) and a data port. The HMI connects a person to a machine, system, or device and allows operators to control and interact with the machine. The data port is a physical interface or connection point that allows for transmission and reception of data between the first computing device (126) and external devices or networks.

As previously stated, the surrogate fuel is formulated in a gas processing facility, and combusted in an engine such that the combustion reaction generates power. Specifically, the chosen composition of chemical elements forming the surrogate fuel are mixed in precise ratios in the gas processing facility to create the surrogate fuel.

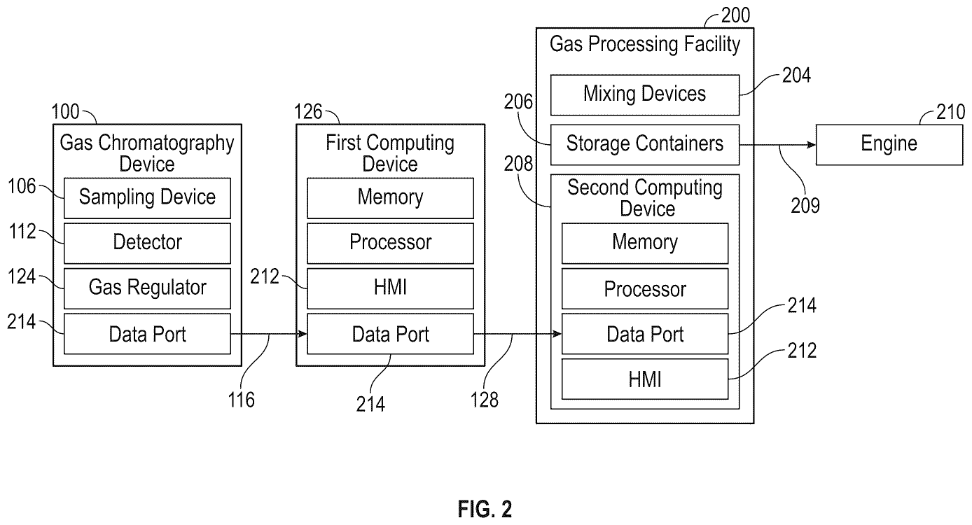

FIG. 2 shows a gas processing facility (200) in accordance with one or more embodiments. The gas processing facility (200) includes a second computing device (208) that comprises or is functionally similar to that of a computer system discussed in greater detail in relation to FIG. 8 and the accompanying description. The second computing device (208) may include a memory and a processor that respectively serve to store and execute computer readable instructions. In addition, the second computing device (208) may include an HMI (212) and a data port (214).

As noted, a data port (214) is a physical interface or connection point that allows for transmission and reception of data between a computing device and external devices or networks. In one or more embodiments, the first computing device (126) and the second computing device (208) are communicably coupled to each other using the second data connection (128). Specifically, the second data connection (128) may connect the data port (214) of the first computing device (126) to the data port (214) of the second computing device (208). As will be described in greater detail below, a plurality of fuel targets defining desired combustion parameters of the surrogate fuel are received by the second computing device (208) and used to determine an optimal surrogate fuel composition, which is then formulated in the gas processing facility (200).

In one or more embodiments, the components, modules, and/or subsystems of the first computing device (126) may communicate wirelessly with a gas processing facility (200) using the second data connection (128). Wireless communication may be facilitated through Radio Frequency Identification (RFID), Near Field Communication (NFC), low-energy Bluetooth, low-energy wireless, low-energy radio protocols, LTE-A, and WiFi-Direct technologies, or other wireless methods, without departing from the scope of this disclosure.

A gas processing facility (200) typically comprises of several components designed to formulate the surrogate fuel. One with ordinary skill in the art will recognize that gas processing facilities (200) may be configured in a variety of ways according to facility-specific needs and applications. As such, the set of sub-processes shown in FIG. 2, and their arrangement, are non-limiting. For the purposes of FIG. 2, components of the gas processing facility (200), may be described according to their function (sub-process) or their mechanical form without undue ambiguity.

As shown in FIG. 2, the gas processing facility (200) may include, at least, mixing devices (204) and storage containers (206). Mixing devices (204) are chemical mixers that are used to ensure that the different hydrocarbons and additives are blended together in precise ratios to achieve the desired physical and chemical characteristics of the surrogate fuel. Examples of mixing devices (204) include static mixers, agitated tank mixers, and inline mixers. Storage containers (206) are used to store raw materials, such as hydrocarbons and additives, that are used to formulate the surrogate fuel. Further, storage containers (206) may also hold intermediate products between different stages of the surrogate fuel production process. In addition, after the surrogate fuel has been formulated and processed, it may be stored in storage containers (206) before it is used for testing or shipped.

A gas processing facility (200) may also include heating and cooling systems to control the temperature during fuel synthesis and processing, which aid in obtaining the desired fuel properties. Distillation columns and membrane separators (or other separation technologies) may be used in the gas processing facility (200) to purify the surrogate fuel by removing unwanted by-products or unreacted components. Further, depending on the type of surrogate fuel being developed, reactors may be used to simulate chemical reactions, such as catalytic or thermal cracking processes, that occur during fuel production.

In one or more embodiments, the gas processing facility (200) may be a research laboratory. Research laboratories often formulate surrogate fuels to study their combustion and emission processes. In other embodiments, the gas processing facility (200) may be a pilot plant. Pilot plants are small-scale industrial facilities where new fuels, including surrogate fuels, may be produced in larger quantities for testing in an engine (210) or other systems (e.g., generators). In another embodiment, the gas processing facility (200) may be a gas station.

In accordance with one or more embodiments, the gas processing facility (200) may be an oil and gas processing facility that formulates the surrogate fuel based on the hydrocarbons (e.g., oil and gas) extracted from drilling a hydrocarbon-bearing formation. The gas processing facility (200) may be part of or contiguous with an oil and gas field. In some embodiments, the gas processing facility (200) may be a hydrocarbon refinery or a chemical plant. Herein, an oil and gas field is broadly defined to comprise of wells which produce at least some oil and/or gas. Hydrocarbon wells typically produce oil, gas, and water in combination. Such facilities are characterized by a plurality of pipes, storage containers (206), pressure vessels, valves, and connections. In particular, the storage containers (206) may hold different types of petroleum and oil products (e.g., crude oil, gasoline, diesel, jet fuel, etc.). In some embodiments, the gas processing facility (200) may be connected to a pipeline network (209) for transporting petroleum products (e.g., the surrogate fuel) to and from refineries, distribution centers, and other terminals. In accordance with one or more embodiments, the pipeline network (209) transports the surrogate fuel from the gas processing facility (200) to an engine (210) for combustion.

As previously stated, the engine (210) and components connected thereto combusts the surrogate fuel formulated by the gas processing facility (200) in a contained manner such that the combustion reaction generates power. The engine (210) may be disposed in a vehicle or may be part of (or may be) a generator setup such that the engine (210) combusts the surrogate fuel to generate electrical power. In accordance with one or more embodiments, the engine (210) may include components not depicted in FIG. 2 such as, but not limited to, an engine block, cylinders, pistons, a crankshaft, fuel injectors, intake and exhaust valves, and an Electronic Control Unit (ECU). One with ordinary skill in the art will appreciate that an engine (210) may include other components and the fact that they are not enumerated herein does not impose a limit on the present disclosure. It is emphasized that the following discussions of an engine (210) are basic summaries and should not be considered limiting.

In one or more embodiments, the engine (210) may include four cylinders positioned in a linear fashion, commonly referred to as an “inline-four,” “straight-four,” or “I-4” engine. In general, the engine (210) may be configured with any number of cylinders, with non-limiting examples including four, six, eight, or twelve cylinders disposed in various inline or “V” configurations. Each cylinder is sized and shaped to form a containment boundary for the corresponding combustion reaction of each cylinder. As a whole, the combustion reaction of each cylinder is timed such that the combustion reactions occur in sequence.

The engine (210) and components connected thereto combusts the surrogate fuel formulated by the gas processing facility (200) in a contained manner such that the combustion reaction actuates the pistons. The pistons are mechanically coupled to the crankshaft, where the crankshaft serves to couple the combined actuation of the pistons into a single motion. Thus, the crankshaft generates power that drives the engine (210) by converting the reciprocating motion of the pistons into rotary motion.

In some embodiments, the engine (210) is an internal combustion engine in which spark ignition is implemented. In other embodiments, the engine (210) is a gasoline compression ignition (GCI) engine. In such an embodiment, spark assisted compression ignition may be implemented. Spark assisted compression ignition is a method used in GCI engines where a spark plug is employed to initiate combustion when conditions are not sufficient for compression ignition alone. The spark plug may generate an ignition arc, or spark, such that combustion is created by sparking the gasoline-air mixture in the combustion chamber of the engine (210). This allows the engine (210) to maintain consistent ignition by using spark assistance, when necessary, while still operating primarily under compression ignition.

As previously stated, the composition of real fuels may vary depending on crude oil sources, refining processes, and local fuel standards. For example, European diesel fuels are typically ultra-low sulfur diesel, whereas diesel in some developing regions might have higher sulfur levels due to less stringent regulations. As discussed above, compositional variations have an impact on vehicle performance, including fuel spray and combustion. As such, accurate predictive models of fuel composition are desirable for engine design and optimization.

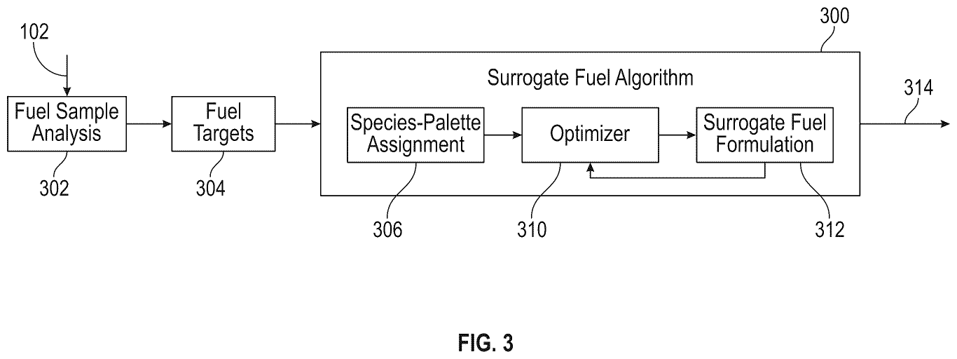

FIG. 3 depicts the process of using a surrogate fuel algorithm (300) to determine the optimal surrogate fuel composition (314) in accordance with one or more embodiments. Initially, fuel sample analysis (302) is performed on a real fuel sample (102) to obtain its fuel properties (e.g., composition). In accordance with one or more embodiments, the fuel sample analysis (302) may be performed using the GC device (100) discussed above with regard to FIG. 1 and the accompanying description. In some embodiments, the fuel sample analysis (302) may be performed using the GC device (100) in conjunction with, or in place of, other experimental techniques, such as, for example, proton nuclear magnetic resonance. In such an embodiment, the molecular structure of the real fuel sample (102) may be also determined.

As discussed, fuel spray behavior is predominantly affected by the physical properties of the liquid phase of the fuel, while the chemical properties of the gas phase of the fuel influence combustion through chemical kinetics. Thus, the liquid sub-surrogate focuses on spray-affecting physical properties, while the gas surrogate closely follows the reactivity characteristics of the real fuel sample (102). Accordingly, it is desirable to have different requirements for liquid and gas sub-surrogates to accurately model the different fuel processes in an engine (210). In accordance with one or more embodiments, the surrogate fuel algorithm (300) receives fuel targets (304) specifying the desired physical and chemical properties of the fuel surrogates. Specifically, the fuel targets (304) may independently describe the target liquid and gas properties of the liquid and gas sub-surrogates, respectively.

In accordance with one or more embodiments, the target physical properties of the liquid sub-surrogate include liquid density and vapor pressure of the real fuel sample (102). The liquid density directly affects the fuel spray's momentum, which, in turn, impacts the spray penetration, air entrainment, liquid break-up, and fuel-air mixing. Vapor pressure has a strong impact on the spray's evaporation process as well as on film formation. Film formation refers to the creation of a thin layer of material (e.g., a polymer or a deposit) on components that come into contact with fuel (e.g., fuel injectors, combustion chambers, fuel lines, etc.) and is typically undesirable since it may lead to decreased performance and increased maintenance. Accordingly, the vapor pressure of the real fuel sample (102) forms one fuel target (304) to match during surrogate modeling. In accordance with one or more embodiments, the vapor pressure may be evaluated at room temperature. One with ordinary skill in the art will recognize that the vapor pressure may be evaluated at any temperature without departing from the scope of this disclosure.

In accordance with one or more embodiments, the target chemical properties of the gas sub-surrogate include molar element ratios (e.g., hydrogen to carbon (H/C) and oxygen to carbon (O/C) ratios) and octane ratings (e.g., a Motor Octane Number (MON) and a Research Octane Number (RON)) of the real fuel sample (102). Fuel-air stoichiometry is a significant combustion metric and heavily depends on the molar element ratio of the fuel. As such, the gas surrogate should represent exact fuel-air equivalence ratio as the real fuel sample (102), which is beneficial for keeping accurate H/C and O/C ratios. Further, octane ratings (e.g., MON and RON) are significant combustion indicators that strongly correlate to the fuel's auto-ignition tendency. Therefore, gas surrogates that match octane ratings of the real fuel sample (102) may accurately predict compression-ignition combustion and abnormal spark-ignition combustion.

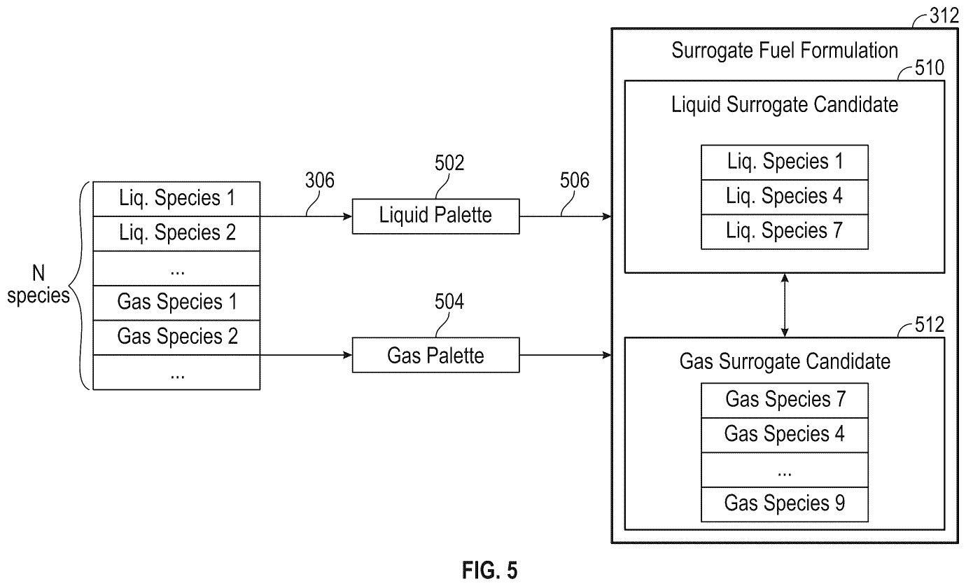

Keeping with FIG. 3, in a species-palette assignment (306) process, liquid and gas fuel species are assigned a palette based on the physical and chemical properties of the real fuel sample (102). Specifically, liquid species are assigned a liquid palette, which is used to determinate the liquid sub-surrogate, and gas fuel species are assigned a gas palette, which is used to determine the gas sub-surrogate. The species-palette assignment (306) process is described in greater detail in relation to FIG. 5 and the accompanying description.

In accordance with one or more embodiments, the liquid palette comprises 10 species, among which at least one species has a boiling point lower than T10 of the real fuel sample (102) and at least one species has a boiling point higher than T90 of the real fuel sample (102). T10 refers to the temperature at which 10% fuel has evaporated during a distillation test. Accordingly, if at least one species of the liquid palette has a boiling point lower than T10, the surrogate fuel contains volatile components that evaporate at temperatures lower than the temperature at which 10% of the real fuel sample (102) evaporates. T10 impacts, for example, the cold start performance of an engine, the risk of vapor lock in fuel systems, and the combustion characteristics. Similarly, T90 refers to the temperature at which 90% of the fuel has evaporated during a distillation test. Thus, having at least one species with a boiling point higher than T90 in the liquid palette implies that some components of the surrogate fuel are heavier and require higher temperatures to evaporate than the temperature at which 90% of the fuel has already evaporated in the real fuel sample (102). T90 adversely affects, for example, combustion quality, engine deposits, and fuel economy.

In accordance with one or more embodiments, the gas palette includes primary reference fuels (PRF), toluene reference fuels (TRF), ethanol (PRF-E) and toluene (TRF-E). PRF are binary mixtures of n-Heptane (a low-octane fuel) and iso-octane (a high-octane fuel). By mixing these two fuels in varying proportions, a range of octane numbers may be created. Further, TRF typically comprise of iso-octane (2,2,4-Trimethylpentane), n-Heptane, and toluene. Toluene is used as a reference fuel because of its high octane rating and its ability to represent the aromatic components of real fuel samples (102). Ethanol (PRF-E) and toluene (TRF-E) expand the applicable ranges.

Keeping with FIG. 3, in accordance with one or more embodiments, an optimizer (310) solves for the optimal combination of palette species that optimize the fuel targets (304). That is, the goal of the optimizer (310) is to alter the combination of palette species to promote similarity between the physical and chemical properties of the surrogate fuel and the physical and chemical properties of the real fuel sample (102) (i.e., fuel targets (304)). Mathematically, optimization takes the following form:

$${arg min}_{{S}_{1}} O \qquad ( 1 )$$

$$subject to : process constraints$$

where the quantity O represents an objective function, although other names such as “error function,” “misfit function,” “loss function,” “cost function,” etc., are commonly employed. The optimization process may iteratively adjust the surrogate fuel formulation (312) until a user-defined termination criterion is met. In one or more embodiments, this iterative process may involve changing or updating the ratios of components, changing or updating palettes, or modifying the optimization parameters until the desired properties of the surrogate fuel are achieved. Once the termination criterion is satisfied, the output of the optimizer (310) is the surrogate fuel composition (314). In some embodiments, the combination of palette species may be a linear combination. In other embodiments, the combination of palette species may be a non-linear combination.

Many types of objective functions are available, such as the mean-squared-error function. However, the general characteristic of an objective function is that it provides a numerical evaluation of the similarity between the optimizer (310) output (i.e., the surrogate fuel composition (314) and associated physical and chemical properties) and the fuel targets (304) (i.e., the physical and chemical properties of the real fuel). Further, in EQ. 1, the set (or sets) of palette species is denoted as S₁. Thus, the objective function is used to guide changes made to the combination of palette species. The optimizer (310) minimizes the objective function (O) over the combination of palette species and outputs a surrogate fuel composition (314).

One with ordinary skill in the art will appreciate that maximization and minimization may be made equivalent through simple techniques such as negation. As such, the choice to represent the optimization as a minimization as shown in EQ. 1 does not limit the scope of the present disclosure. Whether done through minimization or maximization, the optimizer (310) identifies one or more surrogate fuel compositions (314) such that its physical and chemical properties closely match those of the real fuel sample (102).

The surrogate fuel algorithm (300) may implement an iterative solver that implements a repeating algorithmic procedure, where the algorithmic procedure includes a predetermined criterion. In some embodiments, the predetermined criterion is a termination criterion. Common termination criteria include, for example, reaching a fixed number of iterations, a diminishing learning rate, noting no appreciable change in the loss function between iterations, and reaching a specified performance metric as evaluated on a data set. For example, the surrogate fuel algorithm (300) may terminate after reaching a fixed number of iterations, or alternatively once a valid surrogate fuel is identified that satisfies the fuel targets (304). Once the termination criterion is satisfied, the surrogate fuel algorithm (300) returns multiple surrogate fuel candidates (e.g., one for each iteration) with their associated surrogate fuel compositions (314).

In other embodiments, the predetermined criterion may require updating the solution (i.e., the surrogate fuel candidate) until reaching a threshold. The threshold may be selected by a user or automatically determined by the surrogate fuel algorithm (300) (e.g., based on past iterations). For example, a user may determine a threshold, and the surrogate fuel candidate may be continuously compared to the threshold. Specifically, at each iteration, a determination may be made as to whether the updated surrogate fuel candidate has converged. The surrogate fuel algorithm (300) may analyze a change in the current simulated surrogate candidate between one or more previous simulated surrogate candidates to determine convergence. Where a determination is made that convergence is not achieved, the surrogate fuel algorithm (300) may perform another iteration. Where a determination is made that the updated surrogate candidate has converged, the surrogate fuel algorithm (300) returns a single and optimal surrogate fuel candidate with its associated surrogate fuel composition (314).

An iterative solver may use a search method, such as a Newton-Raphson method (also called “Newton's method”), a secant method, or a bisection method, to determine a solution to a particular problem or equation. A Newton-Raphson method may include a root-finding algorithm that produces successively better approximations to the roots of a real-valued function. A secant method may include a root-finding algorithm that uses a succession of roots of secant lines to better approximate a root of a real-valued function. A bisection method may include a root-finding algorithm applicable to various continuous functions that has two known values with opposite signs. The bisection method may repeatedly bisect an interval defined by these known values and then select a subinterval in which a continuous function changes signs (i.e., that contains a root). One with ordinary skill in the art will recognize that any type of search methods and techniques may be employed without departing from the scope of this disclosure. For example, if the Newton-Raphson method or the bisection method have difficulties solving the surrogate fuel candidates, other techniques may be employed.

A gas processing facility (200) may be subject to constraints, such as limits imposed on various devices and sub-processes of a gas processing facility (200). Constraints may arise from, for example, the practical limitations of existing technologies and infrastructure. For instance, in some embodiments, it may be determined that in order to formulate the surrogate fuel determined by the optimizer (310) it may be necessary to modify the equipment in the gas processing facility (200) such that new chemical reactors, blending units, or separation technologies are added for mixing the components. In other embodiments, a gas processing facility (200) may be limited to a daily throughput limit to operate safely. In such an embodiment, throughput, as measured by a given facility device, should not exceed a prescribed value because exceeding this value may lead to mechanical failures or safety risks. In EQ. 1, these constraints are referenced as process constraints. Accordingly, the optimizer (310) cannot select any combination of palette species that cause any portion of the gas processing facility (200) to exceed predefined process constraints. Additional examples of process constraints applied to the optimization may include process integration constraints and emissions regulations constraints.

In accordance with one or more embodiments, the optimizer (310) is a brute-force Monte Carlo search algorithm. A brute-force Monte Carlo algorithm randomly samples palette combinations from the search space (i.e., the space of all possible palette combinations) until an optimal solution is found. When using a brute-force Monte Carlo, a large number of surrogate candidates are evaluated numerically by the optimizer (310) against the fuel targets (304). Evaluation of the surrogate candidates' properties may rely, for example, on open-source or commercial software, or empirical correlation models. In some embodiments, the physical and chemical properties of a surrogate fuel candidate may be estimated based on the known physical and chemical properties of a closely-related surrogate fuel using, for example, interpolation, regression, or extrapolation. A brute-force Monte Carlo algorithm is selected for its ability to enable parallel processing functionality, which advantageously effectuates a faster processing rate compared to linear processing methods. That is, a brute-force Monte Carlo algorithm advantageously may be performed using parallel computing by determining and evaluating multiple surrogate fuel candidates at once, reducing the overall processing time necessary to determine a suitable surrogate fuel candidate that achieves the desired fuel targets.

Consistent with the above, computations for determining fuel surrogates may be performed using various parallel processors to reduce computational runtime, e.g., using a graphical processing unit (GPU)/central processing unit (CPU) hybrid platform. The term “parallel processor” may refer to multiprocessors, individual processors, and computer processors that operate in parallel in a processing operation. By having a CPU and GPU (or other parallel processors) collaborate on deriving fuel surrogates, various computational bottlenecks may be prevented in the data processing. For example, in one or more embodiments, parallel processing may accelerate the optimization process performed by the optimizer (310).

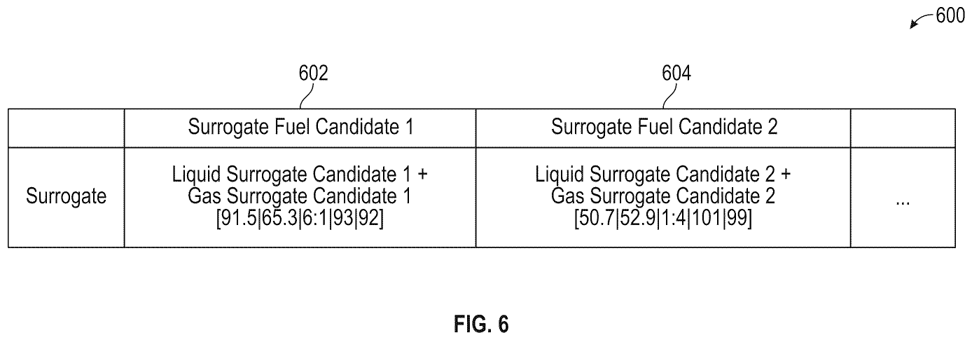

Keeping with FIG. 3, in accordance with one or more embodiments, the output of the surrogate fuel algorithm (300) is the surrogate fuel composition (314). The surrogate fuel composition (314) is a proportional composition of chemical elements forming the surrogate fuel and is determined based on the surrogate fuel formulation (312) as discussed in greater detail below in the instant disclosure with regard to FIG. 6 and the accompanying description. Mass conservation is the primary link between the liquid and gas surrogate candidates. This mass conservation treatment is asymmetric because the liquid palette and the gas palette contain different species.

In accordance with one or more embodiments, the chosen composition of chemical elements forming the surrogate fuel are mixed in precise ratios in a gas processing facility (200) to create the surrogate fuel. Furthermore, the surrogate fuel is tested in specialized combustion chambers or engines (210) to observe its performance under controlled conditions. Combustion parameters such as, for example, ignition delay time, flame luminosity, and emissions may be measured.

While the various blocks in FIG. 3 are presented and described sequentially, one of ordinary skill in the art will appreciate that some or all of the blocks may be executed in different orders, may be combined or omitted, and some or all of the blocks may be executed in parallel.

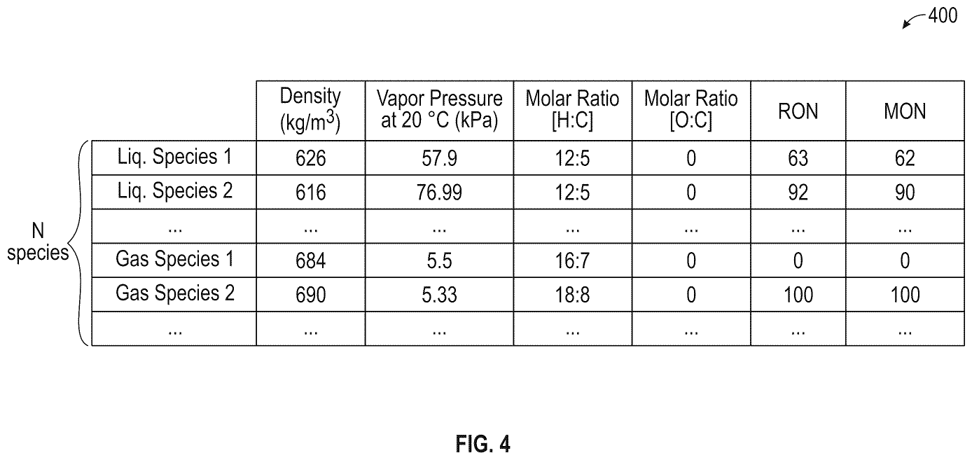

As previously stated, evaluation of the properties of surrogate fuel candidates may rely, for example, on open-source or commercial software, or empirical correlation models. FIG. 4 shows a lookup table (400) with empirically-determined physical and chemical properties of N liquid and gas fuel species in accordance with one or more embodiments. In some embodiments, the physical and chemical properties of the liquid and gas fuel species shown in FIG. 4 may be determined using the GC device (100).

As shown in FIG. 4, the physical properties of the liquid and gas fuel species may include, but are not limited to, density and vapor pressure. In accordance with one or more embodiments, the vapor pressure shown in FIG. 4 in units of kilopascal (kPa) is evaluated at 20 degrees Celsius (° C.). One with ordinary skill in the art will recognize that the vapor pressure may be evaluated at any temperature without departing from the scope of this disclosure. The chemical properties may include, but are not limited to, target molar element ratios (e.g., H/C and O/C ratios) and octane ratings (e.g., MON and RON).

As previously stated, liquid species are assigned to a liquid palette that is used to determinate the liquid sub-surrogate, and gas fuel species are assigned to a gas palette that is used to determine the gas sub-surrogate. FIG. 5 depicts a species-palette assignment (306) process and a palette-surrogate assignment (506) process in accordance with one or more embodiments. Each of the N liquid and gas fuel species shown in FIG. 5 are assigned a liquid palette (502) and a gas palette (504), respectively, in the species-palette assignment (306) process. As noted, a liquid palette (502) comprises at least one component that has a boiling point lower than T10 of the real fuel sample (102) and at least one component has a boiling point higher than T90 of the real fuel sample (102). Further, as previously discussed, the gas palette (504) includes the PRF, TRF, PRF-E, and TRF-E species. The palette-surrogate assignment (506) process, in combination with the optimizer (310), assigns one or more liquid species from the liquid palette (502) to a liquid surrogate candidate and one or more gas species from the gas palette (504) to a gas surrogate candidate. Mass conservation is the primary link between the liquid and gas surrogate candidates. This mass conservation treatment is asymmetric because the liquid palette (502) and the gas palette (504) contain different species. In this regard, the phrase “mass conservation” as utilized herein refers to a liquid surrogate candidate (510) being sourced with a same initial mass as a gas surrogate candidate (512).

By way of a non-limiting example, a surrogate fuel formulation (312) comprises a liquid surrogate candidate (510) for the liquid sub-surrogate (i.e., a liquid surrogate formulation) and a gas surrogate candidate (512) for the gas surrogate (i.e., a gas surrogate formulation), respectively. In the example shown in FIG. 5, the palette-surrogate assignment (506) maps liquid species 1, 4, and 7 from the liquid palette (502) to the liquid surrogate candidate (510), and gas species 7, 4, and 9 from the gas palette (504) to the gas surrogate candidate (512), respectively. In FIG. 5 only one surrogate fuel formulation (312) is shown. However, as previously described, the palette-surrogate assignment (506) process may, in combination with the optimizer (310), generate a large number of surrogate fuel formulations that are numerically evaluated against the fuel targets (304). In such case, parallel processing may be leveraged to accelerate the optimization process performed by the optimizer (310). Therefore, efficient optimization may be advantageously achieved because the evaluation of each surrogate fuel formulation may be computed in parallel using, for example, parallel processors (e.g., a GPU/CPU hybrid platform).

As previously stated, the surrogate fuel composition (314) is a proportional composition of chemical elements forming the surrogate fuel and is determined based on the surrogate fuel formulation (312). FIG. 6 shows surrogate fuel candidates (600) and their associated composition obtained using the surrogate fuel algorithm (300) in accordance with one or more embodiments. As noted, in some embodiments, the surrogate fuel algorithm (300) returns a single and optimal surrogate fuel composition (314). However, in other embodiments, the surrogate fuel algorithm (300) returns multiple surrogate fuel compositions (314). Consequently, in such case, the surrogate fuel algorithm (300) outputs multiple surrogate fuel candidates (600) and their associated surrogate fuel compositions (314).

In the embodiment shown in FIG. 6, the surrogate fuel algorithm (300) outputs at least two surrogate fuel candidates (600). The surrogate fuel candidate 1 (602) comprises liquid surrogate candidate 1 and gas surrogate candidate 1. The surrogate fuel candidate 2 (604) comprises liquid surrogate candidate 2 and gas surrogate candidate 2. In accordance with one or more embodiments, each surrogate fuel candidate in FIG. 6 is shown with its associated physical and chemical properties. As previously stated, the physical and chemical properties of the surrogate fuel candidates (600) are optimized until they closely match those of the real fuel sample (102).

In the embodiment shown in FIG. 6, the surrogate fuel candidate 1 (602) is determined to have a density of 91.5 kg/m³, a vapor pressure of 65.3 kPa at 20° C., a 6:1 ratio of liquid surrogate candidate 1 to gas surrogate candidate 1, a RON of 93, and a MON of 92. The surrogate fuel candidate 2 (604) is determined to have a density of 50.7 kg/m³, a vapor pressure of 52.9 at 20° C., a 1:4 ratio of liquid surrogate candidate 2 to gas surrogate candidate 2, a RON of 101, and a MON of 99. Analytical techniques such as, for example, gas chromatography, mass spectrometry, and infrared spectroscopy may be used to analyze the chemical composition of the selected surrogate fuel and validate that it meets the desired fuel targets (304).

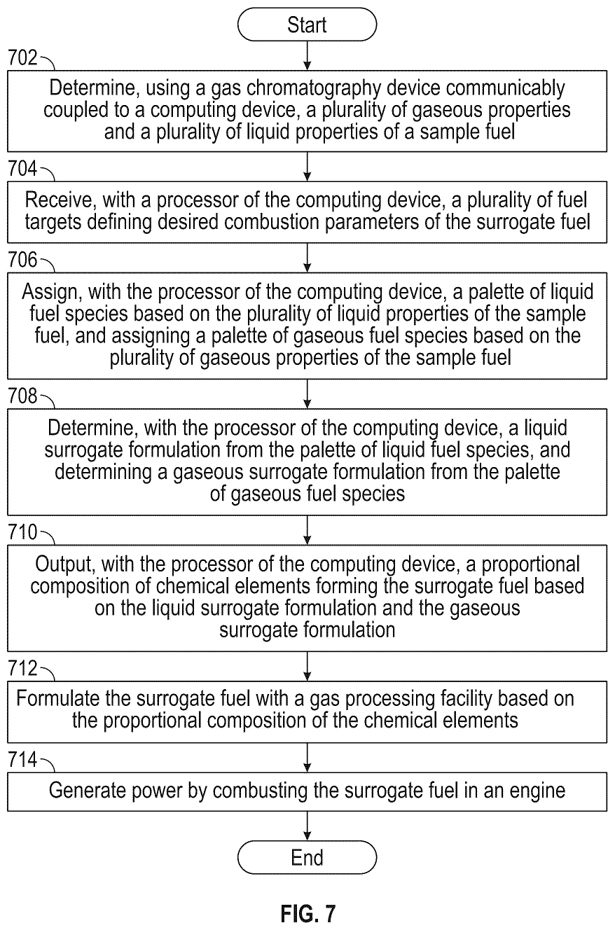

FIG. 7 depicts a method to determine the composition of a surrogate fuel, in accordance with one or more embodiments. It is to be understood that one or more of the steps shown in the flowcharts may be omitted, repeated, and/or performed in a different order than the order shown. Accordingly, the scope disclosed herein should not be considered limited to the specific arrangement of steps shown in the flowcharts.

In Block 702, a plurality of gas properties and a plurality of liquid properties of a sample fuel (e.g., fuel sample (102)) are determined using a GC device (100). A GC device (100) is an analytical instrument used to separate, detect, and analyze the chemical components of compounds that may be vaporized without decomposition. In accordance with one or more embodiments, the signal produced by the detector (112) of the GC device (100) is recorded and analyzed using a first computing device (126). The first computing device (126) comprises or is functionally similar to that of a computer system discussed in greater detail in relation to FIG. 8 and the accompanying description. The first computing device (126) may include a memory and a processor that respectively serve to store and execute computer readable instructions. In addition, the first computing device (126) may include an HMI (212) and a data port (214). The data port is a physical interface or connection point that allows for transmission and reception of data between the first computing device (126) and external devices or networks. In one or more embodiments, the first computing device (126) is communicably coupled to the detector (112) of the GC device (100) using a first data connection (116).

Keeping with Block 702, in one or more embodiments, the components, modules, and/or subsystems of the first computing device (126) may communicate wirelessly with a gas processing facility (200) using a second data connection (128). Wireless communication may be facilitated through RFID, NFC, low-energy Bluetooth, low-energy wireless, low-energy radio protocols, LTE-A, and WiFi-Direct technologies, or other wireless methods, without departing from the scope of this disclosure.

In Block 704, a plurality of fuel targets (304) defining desired combustion parameters of the surrogate fuel are received by a second computing device (208). In some embodiments, the gas processing facility (200) includes the second computing device (208). The second computing device (208) comprises or is functionally similar to that of a computer system discussed in greater detail in relation to FIG. 8 and the accompanying description. The second computing device (208) may include a memory and a processor that respectively serve to store and execute computer readable instructions. In addition, the second computing device (208) may include an HMI (212) and a data port (214). In one or more embodiments, the first computing device (126) and the second computing device (208) are communicably coupled to each other using the second data connection (128).

Keeping with Block 704, in accordance with one or more embodiments, a surrogate fuel algorithm (300) receives the fuel targets (304). The fuel targets (304) specify the desired physical and chemical properties of the fuel surrogates. Specifically, the fuel targets (304) may independently describe the target liquid and gas properties of the liquid and gas sub-surrogates, respectively. In accordance with one or more embodiments, the target physical properties of the liquid sub-surrogate include liquid density and vapor pressure of the real fuel sample (102), and the target chemical properties of the gas sub-surrogate include molar element ratios (e.g., H/C and O/C ratios) and octane ratings (e.g., MON and RON) of the real fuel sample (102).

In Block 706, surrogate liquid and gas fuel species are assigned a palette based on the physical and chemical properties of the real fuel sample (102). Specifically, in the species-palette assignment (306) process, liquid species are assigned a liquid palette (502), which is used to determinate the liquid surrogate candidate (510), and gas fuel species are assigned a gas palette (504), which is used to determine the gas surrogate candidate (512). The liquid palette (502) comprises at least one component that has a boiling point lower than T10 of the real fuel sample (102) and at least one component that has a boiling point higher than T90 of the real fuel sample (102). T10 refers to the temperature at which 10% fuel has evaporated during a distillation test. T90 refers to the temperature at which 90% of the fuel has evaporated during a distillation test. The gas palette (504) includes the PRF, TRF, PRF-E, and TRF-E species.

In Block 708, a palette-surrogate assignment (506) process, in combination with an optimizer (310), determines a surrogate fuel formulation (312) comprising a liquid surrogate candidate (510) for the liquid sub-surrogate (i.e., a liquid surrogate formulation) based on the liquid palette (502) and a gas surrogate candidate (512) for the gas surrogate (i.e., a gas surrogate formulation) based on the gas palette (504). Specifically, the optimizer (310) solves for the optimal combination of palette species that optimize the fuel targets (304). That is, the goal of the optimizer (310) is to alter the combination of palette species to promote similarity between the physical and chemical properties of the surrogate fuel and the physical and chemical properties of the real fuel sample (102) (i.e., fuel targets (304)). In accordance with one or more embodiments, the optimizer (310) is a brute-force Monte Carlo search algorithm. The optimization process may iteratively adjust the surrogate fuel formulation (312) until a user-defined termination criterion is met. In one or more embodiments, this iterative process may involve changing or updating the ratios of components, changing or updating palettes, or modifying the optimization parameters until the desired properties of the surrogate fuel are achieved.

Keeping with Block 708, in accordance with one or more embodiments, the palette-surrogate assignment (506), in combination with the optimizer (310), may generate a large number of surrogate fuel formulations to be evaluated numerically against the fuel targets (304). Parallel processing may be leveraged to accelerate the optimization process performed by the optimizer (310). Therefore, efficient optimization may be advantageously achieved because the evaluation of each surrogate fuel formulation may be computed in parallel using, for example, parallel processors (e.g., a GPU/CPU hybrid platform).

In Block 710, the output of the surrogate fuel algorithm (300) is the surrogate fuel composition (314). The surrogate fuel composition (314) is a proportional composition of chemical elements forming the surrogate fuel and is determined based on the surrogate fuel formulation (312). Mass conservation is the primary link between the liquid and gas surrogate candidates. This mass conservation treatment is asymmetric because the liquid palette (502) and the gas palette (504) contain different species. Specifically, when a certain mass of liquid surrogate evaporates in engine (210) simulations, the exact same mass of gas surrogate is sourced in the simulation domain to maintain mass conservation. In some embodiments, the surrogate fuel algorithm (300) returns a single and optimal surrogate fuel composition (314). However, in other embodiments, the surrogate fuel algorithm (300) returns multiple surrogate fuel compositions (314) candidates.

In Block 712, the surrogate fuel is formulated in a gas processing facility (200). Specifically, the chosen composition of chemical elements forming the surrogate fuel are mixed in precise ratios in the gas processing facility (200) to create the surrogate fuel. The gas processing facility (200) typically comprises several components designed to formulate the surrogate fuel. For instance, the gas processing facility (200) may include, at least, mixing devices (204) and storage containers (206). Mixing devices (204) are chemical mixers that are used to ensure that the different hydrocarbons and additives are blended together in precise ratios to achieve the desired physical and chemical characteristics of the surrogate fuel. Storage containers (206) are used to store raw materials, such as hydrocarbons and additives, that are used to formulate the surrogate fuel and may also hold intermediate products between different stages of the surrogate fuel production process. In addition, after the surrogate fuel has been formulated and processed, it may be stored in storage containers (206) before it is used for testing or shipped. Analytical techniques such as, for example, gas chromatography, mass spectrometry, and infrared spectroscopy may be used to analyze the chemical composition of the surrogate fuel and validate that it meets the desired fuel targets (304).

In Block 714, the surrogate fuel formulated by the gas processing facility (200) is combusted in a contained manner in an engine (210) such that the combustion reaction generates power. The engine (210) may be disposed in a vehicle or may be part of (or may be) a generator setup such that the engine (210) combusts the surrogate fuel to generate electrical power. In some embodiments, the engine (210) may be an internal combustion engine in which spark ignition is implemented. In other embodiments, the engine (210) may be GCI engine. Furthermore, combustion parameters such as, for example, ignition delay times, flame region, and flame luminosity may be measured before, during, or after the combustion reaction.

Keeping with Block 714, in accordance with one or more embodiments, the engine (210) may include, for example, components such as an engine block, cylinders, pistons, a crankshaft, a fuel rail, an air intake line, a compressor, a turbine, an intake manifold, an exhaust manifold, an after-treatment device, an exhaust pipe, and an ECU. The pistons are mechanically coupled to the crankshaft, where the crankshaft serves to couple the combined actuation of the pistons into a single motion. Further, the combustion reaction actuates the pistons. Thus, the crankshaft generates power that drives the engine (210) by converting the reciprocating motion of the pistons into rotary motion.

Overall, the method disclosed herein enables the identification and combustion of a surrogate fuel that meets the fuel target (304) properties while simultaneously minimizing emissions. Consequently, using the formulated surrogate fuel in an engine (210) has tangible benefits, such as reducing the emissions that would have otherwise been produced if the surrogate fuel had not been identified and utilized.

Embodiments disclosed herein may be implemented on a computer system. FIG. 8 is a block diagram of a computer system (802) used to provide computational functionalities associated with described algorithms, methods, functions, processes, flows, and procedures as described in the instant disclosure, according to one or more embodiments. For example, the first computing device (126) communicably coupled to the detector (112) and the second computing device (208) of the gas processing facility (200) may include, or may be, a computer system (802) such as that depicted in FIG. 8. The illustrated computer (802) is intended to encompass any computing device such as a server, desktop computer, laptop/notebook computer, wireless data port, smart phone, personal data assistant (PDA), tablet computing device, one or more processors within these devices, or any other suitable processing device such as an edge computing device, including both physical or virtual instances (or both) of the computing device. An edge computing device is a dedicated computing device that is, typically, physically adjacent to the process or control with which it interacts.

Additionally, the computer (802) may include a computer that includes an input device, such as a keypad, keyboard, touch screen, or other device that may accept user information, and an output device that conveys information associated with the operation of the computer (802), including digital data, visual, or audio information (or a combination of information), or a GUI.

The computer (802) may serve in a role as a client, network component, a server, a database or other persistency, or any other component (or a combination of roles) of a computer system for performing the subject matter described in the instant disclosure. In some implementations, one or more components of the computer (802) may be configured to operate within environments, including cloud-computing-based, local, global, or other environment (or a combination of environments).

At a high level, the computer (802) is an electronic computing device operable to receive, transmit, process, store, or manage data and information associated with the described subject matter. According to some implementations, the computer (802) may also include or be communicably coupled with an application server, e-mail server, web server, caching server, streaming data server, business intelligence (BI) server, or other server (or a combination of servers).

The computer (802) may receive requests over network (830) from a client application (for example, executing on another computer (802) and responding to the received requests by processing the said requests in an appropriate software application. In addition, requests may also be sent to the computer (802) from internal users (for example, from a command console or by other appropriate access method), external or third-parties, other automated applications, as well as any other appropriate entities, individuals, systems, or computers.

Each of the components of the computer (802) may communicate using a system bus (803). In some implementations, any or all of the components of the computer (802), both hardware or software (or a combination of hardware and software), may interface with each other or the interface (804) (or a combination of both) over the system bus (803) using an application programming interface (API) (812) or a service layer (813) (or a combination of the API (812) and service layer (813). The API (812) may include specifications for routines, data structures, and object classes. The API (812) may be either computer-language independent or dependent and refer to a complete interface, a single function, or even a set of APIs. The service layer (813) provides software services to the computer (802) or other components (whether or not illustrated) that are communicably coupled to the computer (802). The functionality of the computer (802) may be accessible for all service consumers using this service layer. Software services, such as those provided by the service layer (813), provide reusable, defined business functionalities through a defined interface. For example, the interface may be software written in JAVA, C++, or other suitable language providing data in extensible markup language (XML) format or another suitable format. While illustrated as an integrated component of the computer (802), alternative implementations may illustrate the API (812) or the service layer (813) as stand-alone components in relation to other components of the computer (802) or other components (whether or not illustrated) that are communicably coupled to the computer (802). Moreover, any or all parts of the API (812) or the service layer (813) may be implemented as child or sub-modules of another software module, enterprise application, or hardware module without departing from the scope of this disclosure.

The computer (802) includes an interface (804). Although illustrated as a single interface (804) in FIG. 8, two or more interfaces (804) may be used according to particular needs, desires, or particular implementations of the computer (802). The interface (804) is used by the computer (802) for communicating with other systems in a distributed environment that are connected to the network (830). Generally, the interface (804) includes logic encoded in software or hardware (or a combination of software and hardware) and operable to communicate with the network (830). More specifically, the interface (804) may include software supporting one or more communication protocols associated with communications such that the network (830) or interface's hardware is operable to communicate physical signals within and outside of the illustrated computer (802).

The computer (802) includes at least one computer processor (805). Although illustrated as a single computer processor (805) in FIG. 8, two or more processors may be used according to particular needs, desires, or particular implementations of the computer (802). Generally, the computer processor (805) executes instructions and manipulates data to perform the operations of the computer (802) and any algorithms, methods, functions, processes, flows, and procedures as described in the instant disclosure.

The computer (802) also includes a memory (806) that holds data for the computer (802) or other components (or a combination of both) that may be connected to the network (830). The memory may be a non-transitory computer readable medium such as a Hard Disk Drive (HDD) or a Solid State Drive (SSD). For example, memory (806) may be a database storing data consistent with this disclosure. Although illustrated as a single memory (806) in FIG. 8, two or more memories may be used according to particular needs, desires, or particular implementations of the computer (802) and the described functionality. While memory (806) is illustrated as an integral component of the computer (802), in alternative implementations, memory (806) may be external to the computer (802).

The application (807) is an algorithmic software engine providing functionality according to particular needs, desires, or particular implementations of the computer (802), particularly with respect to functionality described in this disclosure. For example, application (807) may serve as one or more components, modules, applications, etc. Further, although illustrated as a single application (807), the application (807) may be implemented as multiple applications (807) on the computer (802). In addition, although illustrated as integral to the computer (802), in alternative implementations, the application (807) may be external to the computer (802).

There may be any number of computers (802) associated with, or external to, a computer system containing computer (802), wherein each computer (802) communicates over network (830). Further, the term “client,” “user,” and other appropriate terminology may be used interchangeably as appropriate without departing from the scope of this disclosure. Moreover, this disclosure contemplates that many users may use one computer (802), or that one user may use multiple computers (802).

Embodiments of the present disclosure may provide at least one of the following advantages. As previously stated, the fuel targets (304) may independently describe the target liquid and gas properties of the liquid and gas sub-surrogates, respectively. Specifically, the target physical properties of the liquid sub-surrogate in the present disclosure include the liquid density and the vapor pressure of the real fuel sample (102), and the target chemical properties of the gas sub-surrogate include molar element ratios (e.g., H/C and O/C ratios) and octane ratings (e.g., MON and RON) of the real fuel sample (102). This selection of these target physical and chemical properties, which are advantageously decoupled from each other in the present disclosure, may provide an improved methodology to obtain surrogate fuel formulations (312) suitable to predict the effects of compositional variations that affect fuel spray and combustion. Once decoupled, each sub-surrogate is only responsible for a subset of target properties with less constraints, thus less compromise is necessary during the formulation.

Another benefit of one or more embodiments disclosed herein is the use of an optimizer algorithm, such as, for example, a brute-force Monte Carlo search algorithm, and the use of asymmetric mass conservation for linking the liquid and gas sub-surrogates. Further, as noted, embodiments disclosed herein advantageously adopt a liquid palette (502) that comprises 10 species and a gas palette (504) that comprises the PRF, TRF, PRF-E, and TRF-E species. This choice of a low number of palette species for the liquid palette (502) and simple multi-component species for the gas palette (504) may achieve the most balanced benefits across property accuracy and computational cost because combustion kinetic models exist and are well-developed for these species.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed is:

1. A method to determine a composition of a surrogate fuel, the method comprising:
determining, using a gas chromatography device communicably coupled to a computing device, a plurality of gas properties and a plurality of liquid properties of a sample fuel;
receiving, with a processor of the computing device, a plurality of fuel targets defining desired combustion parameters of the surrogate fuel;
assigning, with the processor of the computing device, a palette of liquid fuel species based on the plurality of liquid properties of the sample fuel, and assigning a palette of gas fuel species based on the plurality of gas properties of the sample fuel;
determining, with the processor of the computing device, a liquid surrogate formulation from the palette of liquid fuel species, and determining a gas surrogate formulation from the palette of gas fuel species;
outputting, with the processor of the computing device, a proportional composition of chemical elements forming the surrogate fuel based on the liquid surrogate formulation and the gas surrogate formulation, and
formulating the surrogate fuel with a gas processing facility based on the proportional composition of the chemical elements.

2. The method of claim 1, wherein the plurality of fuel targets of the sample fuel comprises a ratio of a plurality of molar elements comprising hydrogen, carbon, and oxygen.

3. The method of claim 1, wherein the plurality of fuel targets of the sample fuel includes an octane rating comprising a research octane number (RON) and a Motor Octane Number (MON).

4. The method of claim 1, wherein the plurality of fuel targets of the sample fuel comprises a liquid density target value.

5. The method of claim 1, wherein the plurality of fuel targets of the sample fuel comprises a liquid volatility target value.

6. The method of claim 1, wherein the palette of liquid fuel species comprises a first liquid species having a boiling point temperature with a value of 10% of a boiling point temperature of the sample fuel.

7. The method of claim 1, wherein the palette of liquid fuel species comprises a second liquid species having a boiling point temperature with a value of 90% of a boiling point temperature of the sample fuel.

8. The method of claim 1, wherein the palette of gas fuel species comprises primary reference fuels (PRF), toluene reference fuels (TRF), ethanol (PRF-E), toluene (TRF-E), or a combination thereof.

9. The method of claim 1, wherein the plurality of gas properties and the plurality of liquid properties of the sample fuel are determined using a flame ionization detection process, a thermal conductivity detection process, or a mass spectrometer detection process.

10. The method of claim 1, wherein a liquid mass value determined for the liquid surrogate formulation is equivalent to a gas mass value determined for the gas surrogate formulation.

11. A system to determine a composition of a surrogate fuel, the system comprising:
a computing device comprising a processor configured to execute instructions and a memory configured to store the instructions;
a gas chromatography device communicably coupled to the computing device and configured to determine a plurality of gas properties and a plurality of liquid properties of a sample fuel,
wherein the processor is configured to:
receive a plurality of fuel targets defining desired combustion parameters of the surrogate fuel;
assign a palette of liquid fuel species based on the plurality of liquid properties of the sample fuel, and assign a palette of gas fuel species based on the plurality of gas properties of the sample fuel;
determine a liquid surrogate formulation from the palette of liquid fuel species, and determine a gas surrogate formulation from the palette of gas fuel species, and
output a proportional composition of chemical elements forming the surrogate fuel based on the liquid surrogate formulation and the gas surrogate formulation, and
a gas processing facility configured to formulate the surrogate fuel based on the proportional composition of the chemical elements.

12. The system of claim 11, wherein the plurality of fuel targets of the sample fuel comprises a ratio of a plurality of molar elements comprising hydrogen, carbon, and oxygen.

13. The system of claim 11, wherein the plurality of fuel targets of the sample fuel includes an octane rating comprising a Research Octane Number (RON) and a Motor Octane Number (MON).

14. The system of claim 11, wherein the plurality of fuel targets of the sample fuel comprises a liquid density target value.

15. The system of claim 11, wherein the plurality of liquid properties of the sample fuel comprises a liquid volatility target value.

16. The system of claim 11, wherein the palette of liquid fuel species comprises a first liquid species having a boiling point temperature with a value of 10% of a boiling point temperature of the sample fuel.

17. The system of claim 11, wherein the palette of liquid fuel species comprises a second liquid species having a boiling point temperature with a value of 90% of a boiling point temperature of the sample fuel.

18. The system of claim 11, wherein the palette of gas fuel species comprises primary reference fuels (PRF), toluene reference fuels (TRF), ethanol (PRF-E), toluene (TRF-E), or a combination thereof.

19. The system of claim 11, wherein the gas chromatography device comprises a detector including a flame ionization detector, a thermal conductivity detector, or a mass spectrometer, wherein the detector is configured to determine the plurality of gas properties and the plurality of liquid properties of the sample fuel.

20. The system of claim 11, wherein a liquid mass value determined for the liquid surrogate formulation is equivalent to a gas mass value determined for the gas surrogate formulation.
